(19) United States
(12) Patent Application Publication
(10) Pub. No.: US 2011/0118678 A1
(43) Pub. Date: May 19, 2011

(54) Condensed eye dropper alignment apparatus and method for using same

(76) Inventors: Jeffrey Rehkemper, Chicago, IL (US); David T. Tse, Weston, FL (US); Steven Rehkemper, Chicago, IL (US); Jay Tapper, Wayne, PA (US); Brad D. Simons, Palm Beach Gardens, FL (US)

(21) Appl. No.: 12/914,577

(22) Filed: Oct. 28, 2010

Related U.S. Application Data
(63) Continuation-in-part of application No. 12/608,812, filed on Oct. 29, 2009.
(60) Provisional application No. 61/254,093, filed on Oct. 22, 2009.
(60) Provisional application No. 61/256,189, filed on Oct. 29, 2009.
(60) Provisional application No. 61/259,741, filed on Nov. 10, 2009.
(60) Provisional application No. 61/294,316, filed on Jan. 12, 2010.

(51) Int. Cl. A61F 9/00

(52) U.S. Cl. 604/290; 604/300

(57) ABSTRACT

The present application discloses a new and novel system and apparatus for improving the accuracy with which a person may apply eye drops to their own eye. The apparatus holds a bottle of eye drops at a fixed and certain distance above the eye, beside the nose and below the forehead brow such that the user may easily apply eye drops consistently to one particular portion of their own eye. The apparatus sits on the nasion of the nose using a nasal bridge device and also rests on two pillars that are placed on the user's forehead. Used in such a manner, the apparatus facilitates one-handed eye drop application, facilitates both non-visual alignment and oblique alignment, and reduces reflective blinking.

This application is a continuation-in-part of U.S. patent application Ser. No. 12/608,812, filed Oct. 29, 2009, which claims the priority benefit of U.S. application Ser. No. 61/254,093, filed Oct. 22, 2009, and also claims the priority benefit of U.S. application Ser. No. 61/256,189, filed Oct. 29, 2009, U.S. application Ser. No. 61/259,741, filed Nov. 10, 2009 and U.S. application Ser. No. 61/294,316, filed Jan. 12, 2010, the disclosures of which are incorporated herein by reference in their entirety.

### FIELD

The present application provides a placement, stabilization, locating and positioning device for aligning an eye dropper, and a method for improving eye drop application using the device. The device facilitates an alignment wherein the eye drop bottle and bottom of an eye drop supply are disposed relative to a selected eye location, so that the eye drops are applied off of a visual axis drawn from the retina and through the center of the pupil. This enables both one-handed and two-handed dispensing of eye drops from the eye dropper in a manner that facilitates the non-visual alignment of an eye drop device and precludes reflexive blinking by assuring the selected alignment between the eye dropper and the eye, freeing the user from adjusting alignment and allowing the user to focus exclusively on dispensing the liquid from the eye dropper. Dual alignment wells correspond to both eyes so that a single placement of the device provides an alignment without having to reposition the device for each eye. By optimizing eye drop delivery, this device will minimize medication wastage. Contamination is avoided by shielding the eye dropper nozzle from a user's eyes and eyelashes.

### BACKGROUND

Currently in the art there is a need for an eye dropper application apparatus which automatically locates an eye drop dispenser and provides stability in all three directional axes of movement; up and down the height of the eye, side to side over the width of the eye and in and out over and above the surface of the eye.

Presently in the art, apparatus using a nasal bridge saddle as a support do not provide for control about all three axes, are not particularly safe and lack the ability to accommodate both eyes.

Furthermore, there is no apparatus that provides a safer and more functionally reliable device that will preclude reflexive blinking before a drop makes contact with the eye by a user.

Reflexive blinking is influenced by visual clues and tactile sensation. If an object suddenly flies toward the eye, the eyelids will reflexively close at high speed and the head flinches—a reflex to a visual threat without any volitional control. On the other hand, if a blast of air from a jet hits the eye, the eye will reflexively blink even though it cannot see the air coming but the cornea feels the air because of tactile sensation. The design of an eye drop delivery system should be aimed at minimizing contributory factors to reflexive blinking. The user should not see the drop coming and sensation should be minimal upon eye drop contact with the eye. By not seeing the approaching eye drop, the fear of a pending strike is eliminated and thus minimizing reflexive blinking. When the eye drop does make contact with a much less sensitive caruncle, the anxiety for future application is removed, thus enhancing medication compliance.

Gibilsco (U.S. Pat. No. 4,257,417) exists in the art as a nose supported eye dropper holder. However, this apparatus only holds the bottle stable in a side-to-side direction. The holder does not produce any resistance to motion in a towards and away from the eye axis of motion, also called height precision locking. The motion of the eye dropper moving in such a manner may lead to the eye being impacted by the eye dropper and such an impact may be a source of infection or may scratch the surface of the eye. Furthermore, Gibilsco does not contain any discernable means by which up and down motion across the height of the eye may be presented, also called a stability locking mechanism. The lack of up and down stability, combined with the lack of in and out stability could even result in lack of accuracy in the side to side eye direction, as the combination of pulling the eye dropper too high up could also overshoot the iris.

Wood (U.S. Pat. No. 2,676,592) exists in the art as a nose support guide for eye droppers. However, this apparatus only positions the eye dropper over the user's eye and prevents motion only in a side-to-side axis. There is no element within this device that stops the eye dropper from moving in or out relative to the surface of the eye, thus the eye dropper can still impact with the surface of the eye. Further, Wood does not use eye pads or any part to prevent an eye dropper from pivoting in an up and down axis of motion across the height of the eye. Thus, Wood only dampens motion in the back and forth across the width of the eye axis of motion.

Campagna (U.S. Pat. No. 3,934,590) exists in the art as a device that attempts to stabilize an eye drop dispenser through use of a tripod with a pillar that rests on the forehead and another pillar that rests on the cheek. However, it does not provide a precise locator. For such a device to be used on both eyes, the device must be used on one eye and then rotated or flipped for use on the contralateral eye, such that the pillar which originally rested on the forehead over the first eye, upon flipping now rests on the cheek below the contralateral eye, and likewise the pillars that originally rested on the cheek below the first eye upon flipping now rests on the forehead above the contralateral eye. Thus, for the device to be flipped and align properly, the user's forehead and cheek must be of the same distance from the bottle since the pillars are not adjustable. Equally symmetric forehead and cheek dimensions are not typical or prevalent among humans, thus the device is not precise and cannot be flipped for a large portion of the population. Furthermore, the pillars are narrow, relatively pointed shafts and with fixed dimensions. Many users may be reluctant to place two pointed shafts near their eyes for fear of sustaining eye injury. Fear is a major barrier to acceptance and compliance. Because the non-adjustable pillars are of fixed dimension and angle of projection, two pillars may not rest on stable bone but instead on the eyelids. Further still, the device only applies drops to one eye at a time and does not allow the administration of drops to both eyes without reposition and realignment of the device. The Campagna design requires three disparate surfaces for device stabilization—nose, forehead and cheek. In contrast, the subject embodiments have three-point fixation, but only require two surfaces for stabilization—nose and forehead.

There exists a need in the art for an eye dropper holder that first locates precisely and also supports, stabilizes, and holds stationary, an eye dropper in all three of the up-down, side-to-side, and in-out axis, and which may function to accommodate both eyes with a single alignment placement by a user. There is a need for a device that can deliver an eye drop without causing reflexive blinking.

### BRIEF DESCRIPTION OF THE DRAWINGS

FIG. 1A is a perspective drawing of the device in one-handed use.

FIG. 1B is a perspective drawing of the device in two-handed use.

FIG. 2 is a perspective view of the device alone.

FIG. 3 is a view featuring the top of the delivery platform of the device.

FIG. 4 is a view featuring the bottom of the delivery platform of the device.

FIG. 5 is an edge on top view featuring the pillar supports of the device.

FIG. 6 is an edge on side view featuring the layout of the device.

FIG. 7 is a view of the motion dampening in the in and out axis.

FIG. 8 is a view of the motion dampening in an up and down axis and a side-to-side axis.

FIG. 9 is a close up detailed view of the nasal bridge saddle.

FIG. 10 is a series of views of the nasal bridge.

FIG. 11 illustrates a condenser two delivery well device.

FIG. 12 illustrates delivery wells that are incomplete circles.

FIG. 13 illustrates a single delivery well embodiment.

FIG. 14 illustrates a single delivery well containing an eye drop supply.

FIG. 15 illustrates a disposable embodiment with an internal eye drop supply.

FIG. 16 illustrates an asymmetric nose pads on the nasal bridge.

FIG. 17 illustrates a detachable symmetric nasal bridge.

FIG. 18 illustrates a method of using the eye drop dispenser stabilizer.

FIG. 19 is an embodiment with eyelash guards.

FIG. 20 is an embodiment with eyelash guards.

FIG. 21 is a bottom view of the eyelash guards.

FIG. 22 is a close up view of the eyelash guards.

FIGS. 23A-23D illustrate a method set of user instructions.

### DETAILED DESCRIPTION

The present application overcomes limitations in the prior art by presenting a new and novel way to enable a person to align and stabilize an eye dropper while applying eye drops to their own eyes in a softer and easier manner. The present application is self locating on the user's face and prevents motion in three directional axes, namely, up and down the face, back and forth across the face and in and out toward the face. The present application also provides two holes for simultaneous eye drop application to both eyes or one eye at a time without moving the platform. The present application also contains a guard to shield the eye dropper nozzle that prevents the eyelash from striking and contaminating the tip of the eye dropper.

The desirable outcome is to optimize precision location of an eye drop application and to minimize wastage. The device offers the advantage of facilitating non-visual alignment of the eye dropper with the eyes, because the device merely has to be placed on the user's face such that the lower nose based support falls on the nasion, where the face and the upper nose join the lower forehead, and which causes the device to be automatically aligned without necessitating any visual input needed to perform alignment. This is a significant distinction because the eyes into which the drops are being placed are the user's visual input sense. Since it is not feasible to put drops into eyes and see at the same time, the ability to perform alignment of an eye dropper to ensure drop delivery without visual input is a feature of the present application. The present application also optimizes efficacy to obtain maximum therapeutic benefit and focused, precise delivery of artificial tears or medication.

One cannot see the eye drop coming because the eye drop delivery is “off visual axis” so the user does not see the drop coming and therefore avoids reflexive blinking. The eye drop dispenser locating apparatus is designed to deliver over the caruncle—a landmark medial to the visual axis.

The present application is usable on both eyes, either singly or simultaneously without flipping the device and does not require a user to place potentially pointed shafts near the eye. The present application may also facilitate bilateral sequential administration of eye drops.

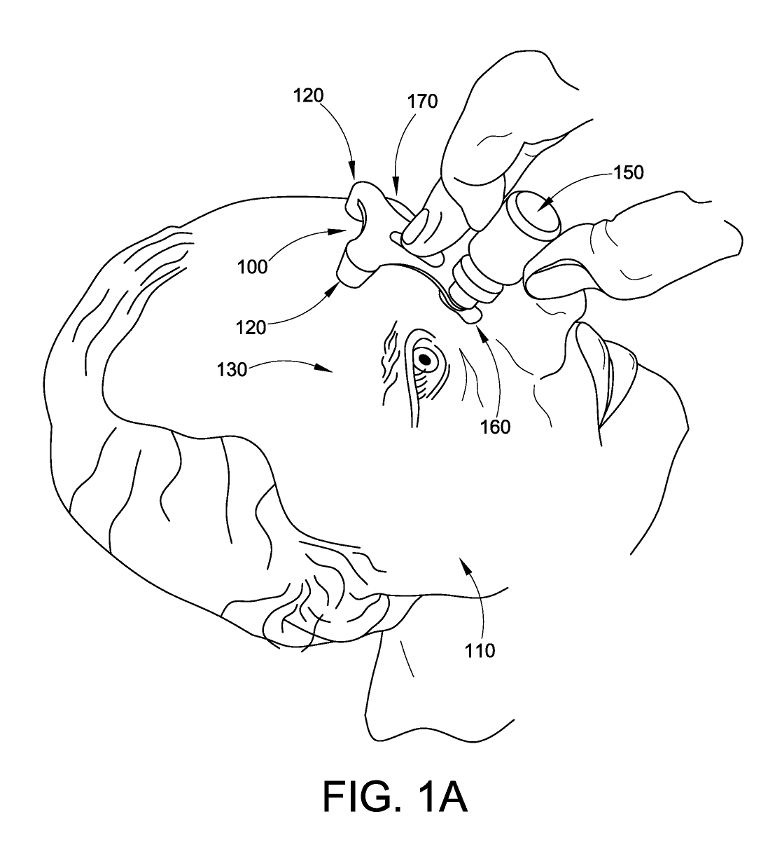

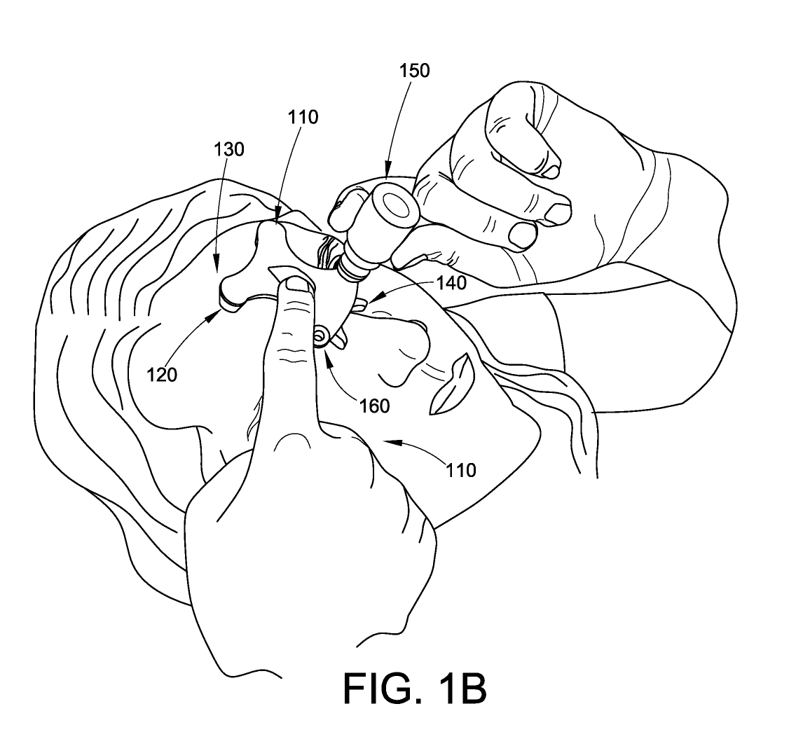

Referring now to FIG. 1A, the eye dropper holder is in use with one-hand. The device 100 is placed such that it rests on the user's face 110 by means of a pair of pillars 120 which rest on a user's forehead 130, and a nasal bridge 140 which rests on a point where the upper portion of the nasal bone intersects the lower forehead. The eye dropper 150 is inserted into the available wells 160, 170. A finger is placed in the center of the eye dropper holder in order to apply a pressure necessary in order to keep the device pressed against the user's face 110. Thus, a single hand may align the device relative to the eyes by also being aligned relative to the nasion.

FIG. 1B shows use of the device with two-hands. The delivery platform may be held in place against the forehead by a finger of either hand applying suitable pressure to the center of the delivery platform. The opposite hand may then squeeze the eye drop dispenser between two fingers, such as the thumb and forefinger. The head should be tilted backward as the eye drops are applied, an optimal angle being about 60 degrees, from the horizon to an axis running through the user's head. For such an axis, the user standing erect and upright, the axis would be at a zero degree inclination. The axis would be parallel to the level horizon upon which the user stands. By tilting the head backward, the angle between the horizon and the axis would increase until the angle reached about 60 degrees. While tilting the head backward would help facilitate the flow of eye drops, the flow of drops could also be facilitated by squeezing the bottle or by a fluid or water jet.

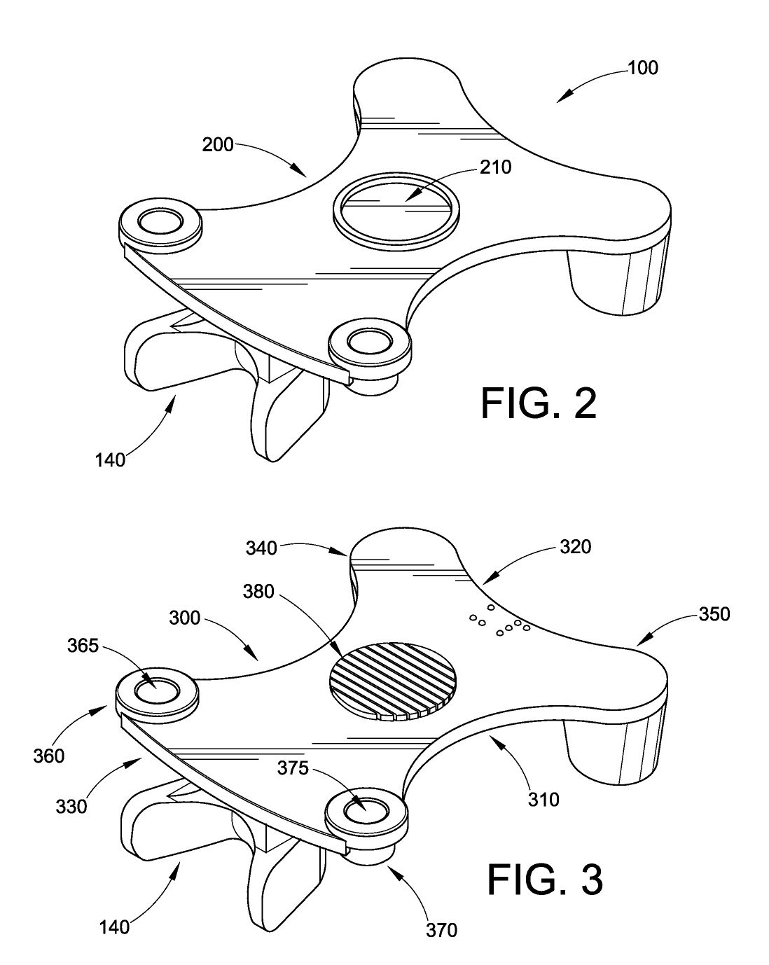

Referring now to FIG. 2, the eye dropper holder 100 comprises one fully assembled unit, which may be made up of separately assembled pieces such as, but not limited to, the delivery platform 200, and the nasal bridge saddle or nasal bridge 140, which may be manufactured as one entire part, or the nasal bridge may be manufactured separate from the delivery platform and assembled together and inserted into the delivery platform 200. The eye dropper holder 100 may be made available with the eye drop container already inserted as an attached bottle, without an eye drop bottle attached, or the eye drops may be packaged in a separate bottle with the eye dropper holder device. The eye dropper holder 100 also contains a holding spot 210 at an upper side to which a user's finger may be placed in order to enable the user to hold the eye dropper holder securely in place during use.

Referring now to FIG. 3, the eye dropper holder 100 is comprised of the delivery platform 200 that is flat and thin. The device could also adequately function if the delivery platform were to be thicker. The delivery platform can be of any geometric shape such as, but not limited to square, rectangle, circle, triangle, and any combination of shapes or polymorphic shape.

The preferred embodiment is a delivery platform 200 which is substantially square, wherein the sides 300, 310 are curved inwardly in an arc shape, with the upper edge 320 being similarly curved with a shallower arc. The lower edge 330 takes the form of an arc that is curving outwardly. The upper edge 320 intersects with the sides 300, 310 for two pronounced semi-circle shaped upper cantilevers 340, 350. The lower edge 330 intersect both the sides 300, 310 by means of smaller semi-circle lower cantilevers 360, 370, each of which contain a delivery well 365, 375.

The intersection of each side edge 300, 310 with the lower edge 330 produces two pronounced semi-circle shaped cantilevers 360, 370. These cantilevers each contain a well 365, 375 located such that the center of the semi-circle shaped cantilever and the center of the well are the same point. The wells must pass all the way through the cantilevers in order for the eye dropper to fit and dispense eye drops. A dispensing nozzle of the eye dropper bottle is closely received in the wells 365, 375. Preferably, the wells comprise a nozzle cylinder extending to a vertical limit such that the terminal end of a received bottle nozzle is shielded against any contaminating contact and a dispensed drop may fall through the cylinder without contacting any portion of the inner well of the cylinder. For perspective, the nasal bridge 140 is also shown.

The delivery wells may extend cylindrically in a direction beneath and perpendicular to the delivery platform, toward the user's eye, to form a nozzle cylinder. When an eye drop dispenser is fitted into the delivery well, the tip of the dispenser is contained within the nozzle cylinder. The cylinder prevents the eyelashes from coming in contact with the eye dropper bottle and prevents contamination of the tip of the eye dropper.

A further embodiment of the delivery well is comprised of an inner cylinder and an outer cylinder in order to prevent contamination of the eye drop dispenser. The inner cylinder is tapered, with the upper portion of the well opening on the upper surface of the delivery platform. This opening possesses the largest diameter of the delivery well. The inner cylinder then tapers and becomes narrower as the depth of the well relative to the upper surface of the delivery platform increases. The inner cylinder receives the tip of an eye drop delivery apparatus, surrounding and encompassing the tip of the eye drop delivery apparatus. The outer cylinder is not tapered and exists outside of the inner cylinder, enclosing the inner cylinder. The outer cylinder serves to prevent the eyelash from touching the tip of the eye drop dispenser. An eyelash of a closing or winking eye will strike the outer cylinder instead of striking the tip of the eye drop dispenser because the inner cylinder guards the eye drop dispenser tip. This serves to prevent contamination of the tip of the eye drop dispenser because the contaminant from the eyelash will fall on and be deposited on the outer cylinder instead of falling on the tip of the eye drop dispenser.

The nozzle cylinder accommodates the top or tip of an eye drop dispenser by one of; screw threads in the nozzle cylinder that accommodate the screw thread of an eye dropper bottle, a Morse taper or any other tapered nozzle cylinder that facilitates a frictional fit of an eye dropper bottle tap, or a sliding locking mechanism. Rigidly fixing the eye drop dispenser in place facilitates one-handed application of eye drops by the user.

The delivery platform may also possess a holding spot 380 on the side opposite to the side from which the previously mentioned pillars extend. The holding spot may be a raised portion, a rough portion, an indentation, a protrusion, on any other means which would facilitate placement of a user's finger in order for the user to apply pressure to keep the eye dropper holder in place upon the user's forehead. When the holding spot is an indentation, the indentation may pass all the way through the thickness of the delivery platform, or only partially recessed into the delivery platform. The indentation may be centered in the middle of the delivery platform, or anywhere between an axis running between the two wells and through to the two pillars. The indentation may also be of a particular shape such as, but not limited to a tear drop, a star, a number, letter or other graphical element. The indentation serves the purpose to facilitate a finger such that a user may apply pressure at this specific point to distribute pressure evenly in order to hold the device firmly to the user's face. The indentation may also contain raised letters, engraving, Braille, or a diagram to convey information to the user.

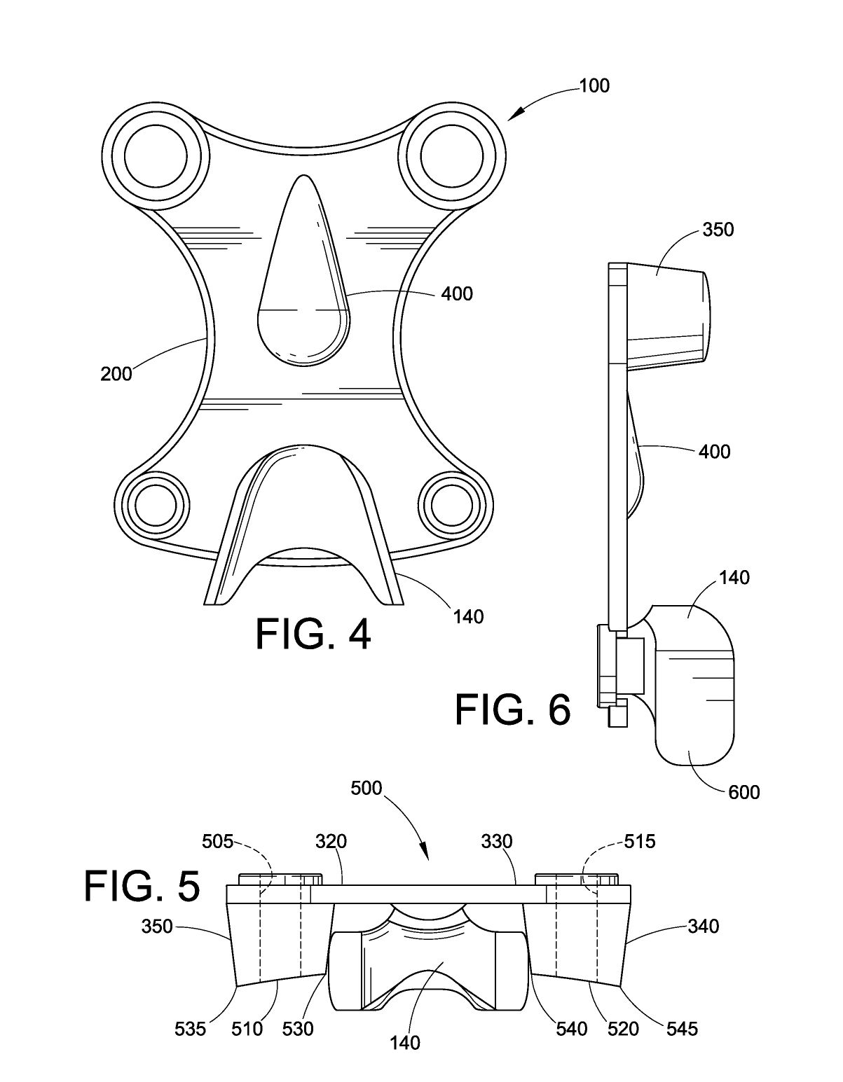

Referring now to FIG. 4, the bottom of the eye dropper holder 100 is comprised of a delivery platform 200 that is flat and thin. When the spot holder is an indentation 400, the indentation may poke outwardly from the bottom of the device. The nasal bridge 140 protrudes from the bottom of the delivery platform 200 and is meant to show the scale relative to the delivery platform.

Referring now to FIG. 5, a view from above 500 shows the pillars or forehead pillars 340, 350 protruding from the upper cantilever 320, wherein each forehead pillar 340, 350, is of any form proportional to support the device for a user, but are shown as substantially circular and hollow 505, 515. The pillars may be solid or partially hollow such that the pillars contain channels. A deluxe embodiment may have a pad within the hollow pillar. The bottom of the pillars 510, 520, may be angled, with the lowest ends 530, 540 pointed toward the inside of the delivery platform and the edges of the pillars 535, 545 facing outward being the longer ends. The two pillars 340, 350 which rest on the user's forehead are short, thick, and placed on the forehead at a height substantially above eye level so as not to provide a threat to eye gouging. The nasal bridge is also shown 140 to scale. The nesting bottle could also attach to the body utilizing a hollow cavity.

Referring now to FIG. 6, the perspective of the length of one pillar 350 and the proportionality of the nasal bridge 140 and the nasal pads 600 are presented. The lengths of the forehead pillar 350 and nasal pad 600 are in proportion to the typical dimensions of an average human being's face. The forehead pillars 340, 350 protrude perpendicular from the delivery platform and also in parallel to the delivery wells 360, 370 within the lower portion of the delivery platform. The underside of the bulge produced by the indentation 400 is also presented.

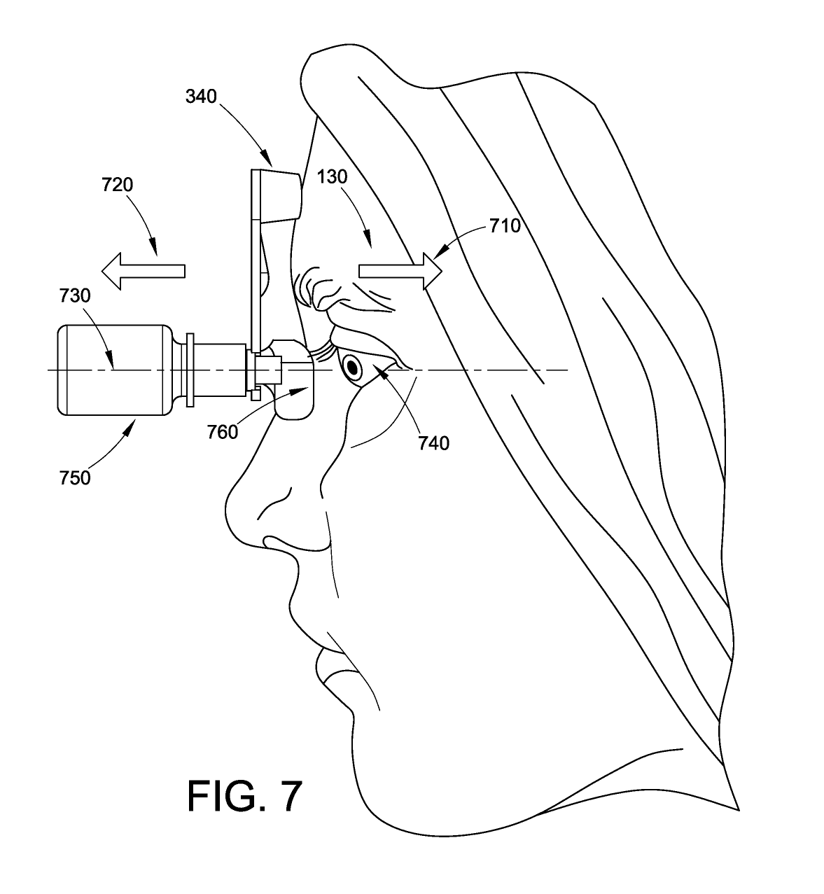

Referring now to FIG. 7, the forehead pillars dampen in and out motion. The forehead pillars 340, 350 as applied to the forehead 130, serve to stabilize the device by dampening motion of the eye dropper coming toward the eye 710, or going away from the eye 720 in an in or out, near or far, towards or away from the eye axis 730 with respect to the surface of the eye 740. This prevents the eye drop dispenser 750 from engaging in contact with the surface of the eye 740 or eyelashes to avoid contamination of the dispenser. The eye drop dispenser 750 can be held in place at a distance 760 suspended above the surface of the eye. The distance is relatively closer than prior art holders to minimize drop velocity at contact but is far enough to prevent infections or scratching that may occur when a contaminated eye drop dispenser comes in contact with the surface of the eye. This height precision locking mechanism is useful to prevent eye dropper impact with the surface of the eyeball.

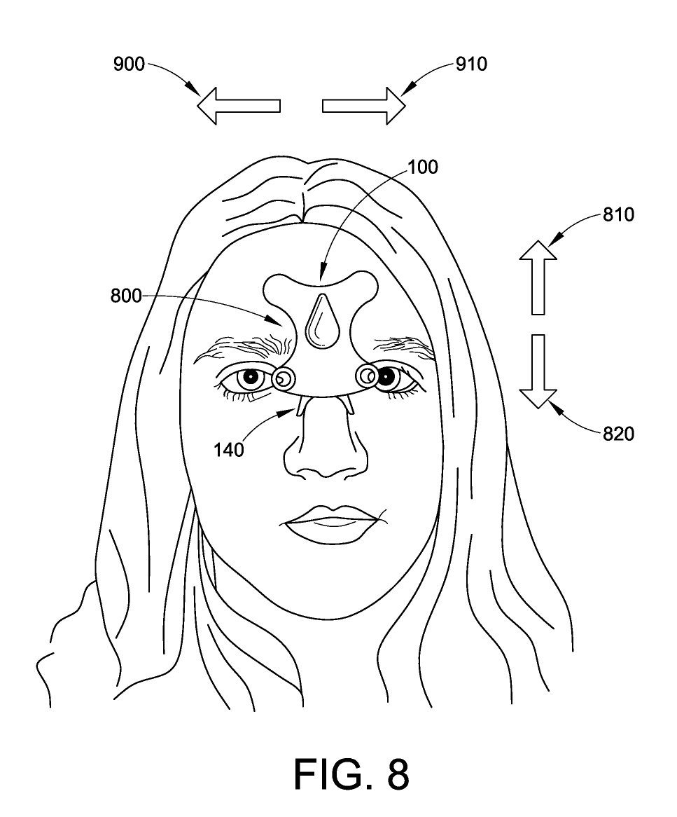

Referring now to FIG. 8, the lower portion that dampens up and down motion is presented. The eye dropper holder 100 is seated on the nasal bridge 140 that rests on the nasion or bridge of the nose 800 at the intersection of the user's upper nose and the user's forehead. The small bridge then tapers down into two cantilever supported slightly diverging nose pads, wherein each nose pad occupies a different side of the user's nose as the bridge rests on the user's nasion. Seating of the bridge of the device upon the user's nasion serves to stabilize the device by dampening motion over the face, from forehead 810 to the chin 820 about the up and down axis such that the eye dropper is held in place over a portion of the eye, such as but not limited to, the iris and is prevented from moving to above or below iris. This stability locking mechanism insures that the drops are applied over the proper location of the eyeball.

The nasal bridge 140 sitting on the nose with pads on the side also dampens side-to-side motion away from the nose 900, and toward the nose 910. The bridge pads serve to stabilize the device by dampening motion in a lateral, side-to-side across the face, from ear-to-ear axis of motion because a nasal pad located on the left side of the nose prevents the eyedropper holder 100 from moving to the left, and the nasal pad on the right side of the nose prevents the eye dropper holder from moving to the right. The pads may be somewhat flexible to accommodate different user nose widths at the nasion. Thus, the eye dropper is held in place over a portion of the eye, such as but not limited to the iris, and is prevented from moving to the side of the iris. This stability locking mechanism insures that the drops are applied over the proper section of the eyeball.

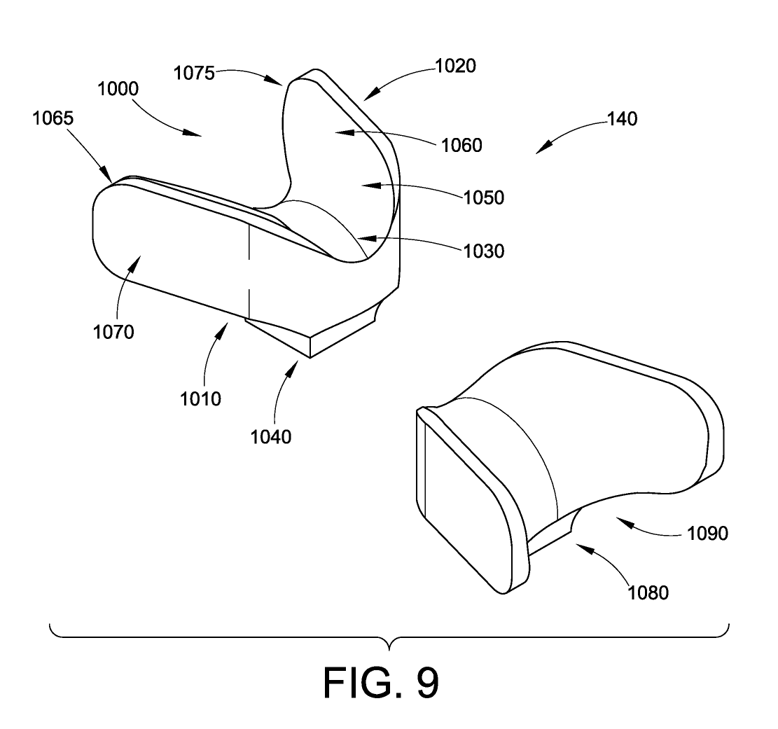

Referring now to FIG. 9, a close up of the contours of the preferred embodiment of the nasal bridge 140 is presented. As observed from below the delivery platform, the nasal bridge 140 is convex shaped and resembles the shape and form of a saddle with intersecting contours, called a first contour and a second contour. The first contour is substantially thinner and flatter than the second contour. The nasal bridge 140 is symmetric about an axis 1000 that divides a left side 1010 of the saddle-shaped sides from the other right side 1020 of the saddle shape. The center contour of the nasal bridge 1030 is smooth and is disposed through a plane substantially parallel to the delivery platform 1040. The center contour gradually slopes upward 1050 in a direction away from the center axis of the delivery platform 1000, gradually ending at a side 1060 which is perpendicular to the center of the nasal bridge 1030 and perpendicular to the delivery platform at the symmetric axis. This surface 1030, 1050 forms a first contour for mating engagement with a first corresponding contour on the face of the user in a direction on a line going from eye-to-eye. The first corresponding contour attaches to the thinnest part of the bridge of the user's nose or the nasion which runs across the width of the nose and slopes down the sides of the nose toward the corners of the eyes. The sides 1060, 1070 extend in a direction away from the delivery platform and proceed in a direction substantially parallel to the symmetric axis 1000. The ends of the nasal bridge sides are rounded in a semi-circular shape 1065, 1075 and resemble pads that occur on the nasal contact in eye glasses. Another view of the center of the nasal bridge 1080 slopes pronouncedly in a downward direction 1090 such that the edge is perpendicular to the bottom of the delivery platform 200. The entirety of the nasal bridge resembles a saddle shape, with the middle or the bridge comprising the seat of the saddle and the perpendicular sides resembling the horns of the saddle shape. This downwardly sloping aspect comprises a second contour, which mates against a second corresponding contour of the face which comprises the nasion of the face located lengthwise running down from the user's forehead and along the length of the user's nose.

Thus, the ventral aspect of the saddle has a convex surface that conforms to the concave anterior surface of the user's nasal bridge. The wing flap on each side of the saddle conforms to a flat descending surface of the nasal bridge, proceeding in the direction of the medial canthus of each eye. Once the device is placed over the nasal bridge, the form-fitted coupling feature of this component will limit the device's displacement in any direction. Superior displacement of the device along the second contour is set and limited by the descending slope of the mid-forehead between the brows. Inferior displacement of the device is limited by the ascending slope of the nose. Lateral movements are limited by the sides of the nose. The two forehead pillars 340, 350 resting on the flat forehead surface confer added stabilization to the device and prevent tilting of the platform when a bottle is inserted into the delivery well.

Thus, the embodiments comprise a device designed to sit precisely on one part of the nose to assure centration of the delivery well over the medial canthus. A visually-impaired person, by tactile clues, can place the nasal bridge saddle 140 of the device over the nose and feel confident in squeezing the bottle. Likewise, a sighted person can deliver the drop in a pitch-dark environment using this device.

In other words, the subject saddle design conforms to the unique contours to the nasal bridge—a perfect fit like a saddle over the back of a horse. The design molds to the nose and has four-point stabilization—descending slope of the nose at 12 o'clock, ascending slope of the nose at 6 o'clock, and the descending slopes on both sides of the nose at a 3 and 9 o'clock hour. On a side profile, it rests firmly on the depressed contour of the nose—a point that aligns precisely in a horizontal axis with the medial canthus or caruncle. By resting on the saddle pillar on the depressed contour of the nose, it automatically aligns the delivery well over the caruncle—a desirable landmark for eye drop placement.

As the width of noses vary greatly within the diverse size and shape of all people and thus potential users, the nasal bridge may vary in dimension to compensate for this diversity. The nasal bridge may be flexible, made of a material that opens up and spreads widely to accommodate thin and thick nasion widths. The nasal bridge may also be rigid, and come in a variety of sizes and shapes, with varying widths. These various sizes may be provided separately from the delivery platforms in order to allow the users to select the proper width bridge and then attach the proper width bridge to the flat main member. Alternatively, the delivery platform may come for purchase with a variety of width sized bridges, each size packaged with or attached to an individual delivery platform. Finally, one delivery platform may come with a plurality of different width bridges in order to accommodate a plurality of users for the same device, such as a family.

In an alternative embodiment, the nasal bridge may be featured without the nose pads. The nasal bridge may also be interchangeable to accommodate different sized noses, or the nose guard may be made of a flexible material to accommodate different nose sizes. The nasal bridge may also slide in a vertical adjustment manner. Such adjustments may be made to customize the device to an individual user of extreme or extraordinary dimensions.

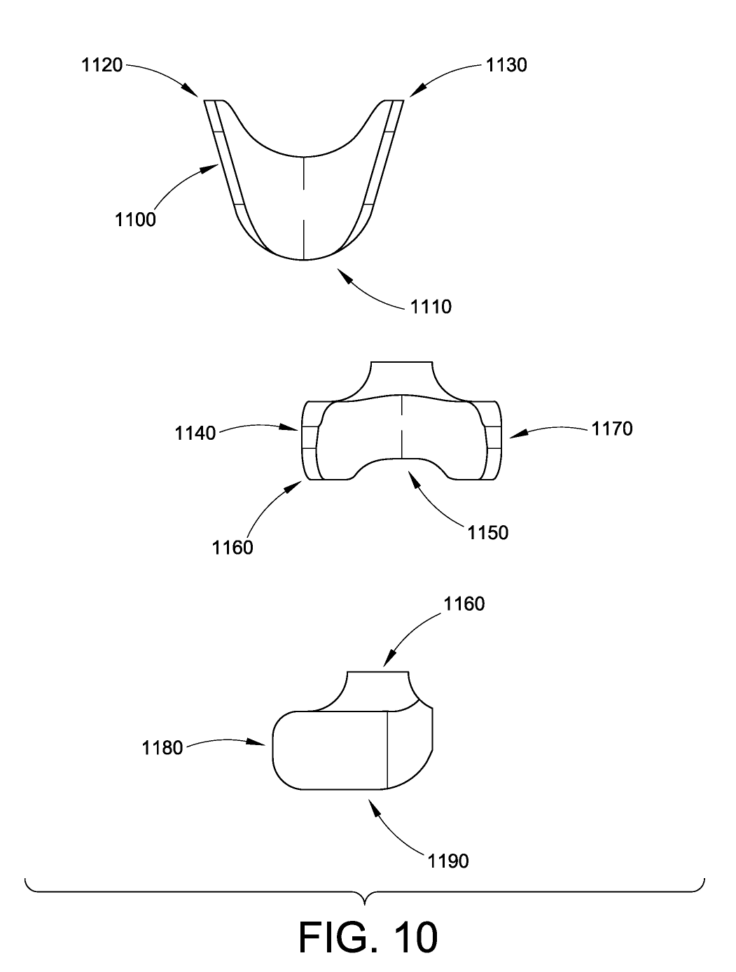

Referring now to FIG. 10, a series of detailed views of the nasal bridge from above or below, 1100, from the front 1140, and from the side 1180 are presented. The above or below view 1100 resembles a crescent shape wherein the center 1110 of the crescent is substantially thicker than is either one end section or other end section 1120. The center section 1110 gently curves and tapers into the end sections 1120, 1130 which form into the eye pads 1065, 1075. The front view 1140 gradually projects outward from a center point 1150 into two equal and symmetric contours 1160, 1170 which become the eye pads 1065, 1075. The side view 1180 illustrates how the center section 1160 forms into a broad cantilever portion 1190 which eventually forms into an eye pad 1065, 1075.

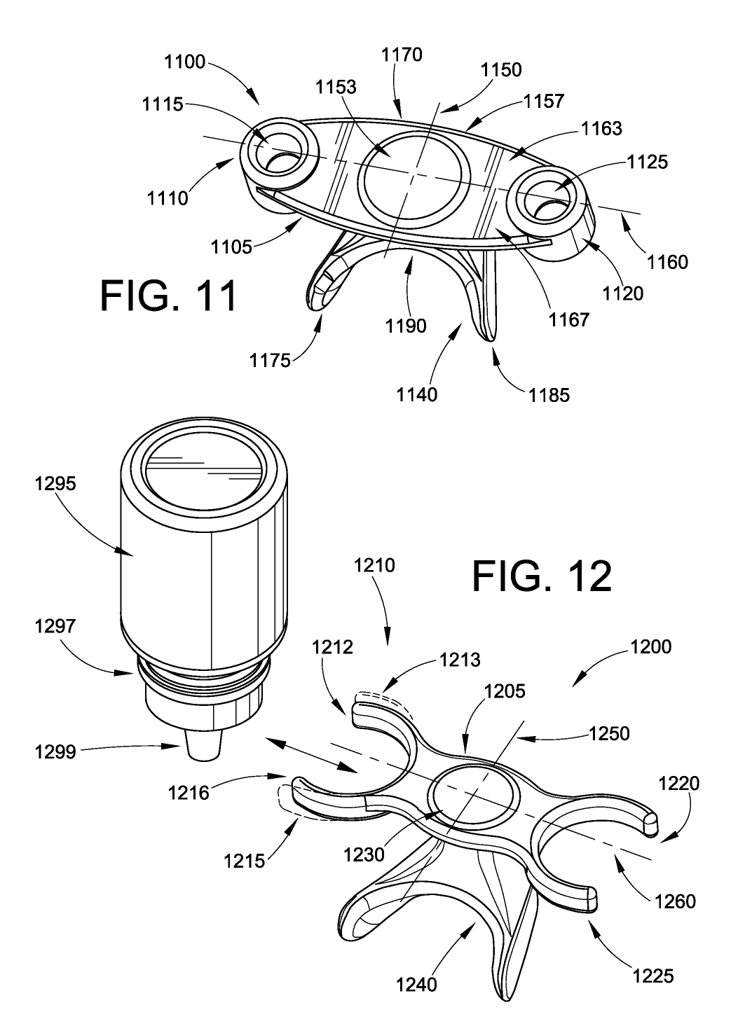

Referring now to FIG. 11, a condensed or abbreviated embodiment of the eye dropper holder 1100 is presented in which the condensed device does not include the stabilizing forehead pillars. The delivery platform 1105 is the shape of an ellipse, with delivery wells 1110, 1120 located at each of the opposing ends of the elliptical platform 1105 and each delivery well 1110, 1120 is the same distance from a centrally located holding spot 1130 on the delivery platform 1105. The nasal bridge 1140 is located beneath and substantially near the center 1130 of the delivery platform 1105. The delivery platform 1105 is substantially symmetric about an axis through the minor axis of the ellipse 1150 and also symmetric about an axis passing through the major axis 1160 of the ellipse.

A plane passing through the minor axis 1150 of the elliptical delivery platform would bisect both the delivery platform 1105 and the nasal bridge 1140 to produce two chiral symmetric halves 1153, 1157 that would be mirror images of each other, with one nasal pad 1175, 1185 on each of the two halves 1153, 1157. A plane passing through the major axis 1160 and passing through the center of the nasal bridge 1140 would produce two non-symmetric halves 1163, 1167 with both of the nasal pads 1175, 1185 on one half and the nasion portion 1190 of the nasal bridge 1140 on the other half.

Other variants or embodiment of the device 1100 may include a delivery platform 1105 in a shape other than an ellipse including, but not limited to, square, rectangle, triangle, or other oblong shape. The delivery platform 1105 also may be asymmetrical to accommodate a user with eyes at different distances from the nose. The interior surface 1115, 1125 of the delivery wells 1110, 1120 may also assume any form previously presented such as tapered, containing a blade, threaded sides and the like. The degree to which the minor axis 1150 intersects the major axis 1160 axis may be perpendicular, but may also be an angle other than perpendicular.

Referring now to FIG. 12, an alternate embodiment of the eye dropper holder 1200 is presented where the delivery platform 1205 contains delivery wells 1210, 1220 that are incomplete circles. The delivery wells 1210, 1220 are missing a section from the circular shape such that the circular delivery wells 1210, 1220 are separated into two different delivery well bracket sides, wherein delivery well 1210 has two bracket sides 1210, 1215 and delivery well 1220 has two bracket sides 1222, 1225. The delivery well brackets 1212, 1215 hold the eye drop dispenser 1290 firmly in place at the side 1295 of an eye drop dispenser 1290, at the neck 1297 of an eye drop dispenser 1290 or at the tip 1299 of an eye drop dispenser 1290. The bracket sides 1212, 1215 are flexible enough to move outwardly 1213, 1216 in order to allow the eye drop dispenser 1290 to pass through the gap between the sides. The bracket sides 1212, 1215 are rigid enough that the tension produced by the sides of the bracket serves to hold the eye drop dispenser 1290 firmly in place.

The device 1200 may contain only one bracket 1210, or may contain a plurality of brackets 1210, 1220 such as the embodiment illustrated. The brackets may be located on directly opposite sides of the delivery platform 1205 such that the delivery platform is symmetric. The dimensions of the plurality of brackets 1210, 1220 may be the same or may be different in order to accommodate different sized eye dropper bottles. The dimensions such as the length of the bracket sides 1212, 1215 may be the same or may be different. The delivery platform 1205 is mounted atop of the nasal bridge 1240 such that a centrally located holding spot 1230 is located above and on an opposite side of the nasal bridge 1240. An axis passing through the minor axis 1250 and bisecting the device may produce two symmetrical mirror image halves, while an axis passing through the major axis 1260 and bisecting the device may produce two asymmetric halves. The dimensions and shape of the delivery wells 1210, 1220 may vary and may be asymmetrical. The degree to which the minor axis 1250 intersects the major axis 1260 axis may be perpendicular, but may also be an angle other than perpendicular.

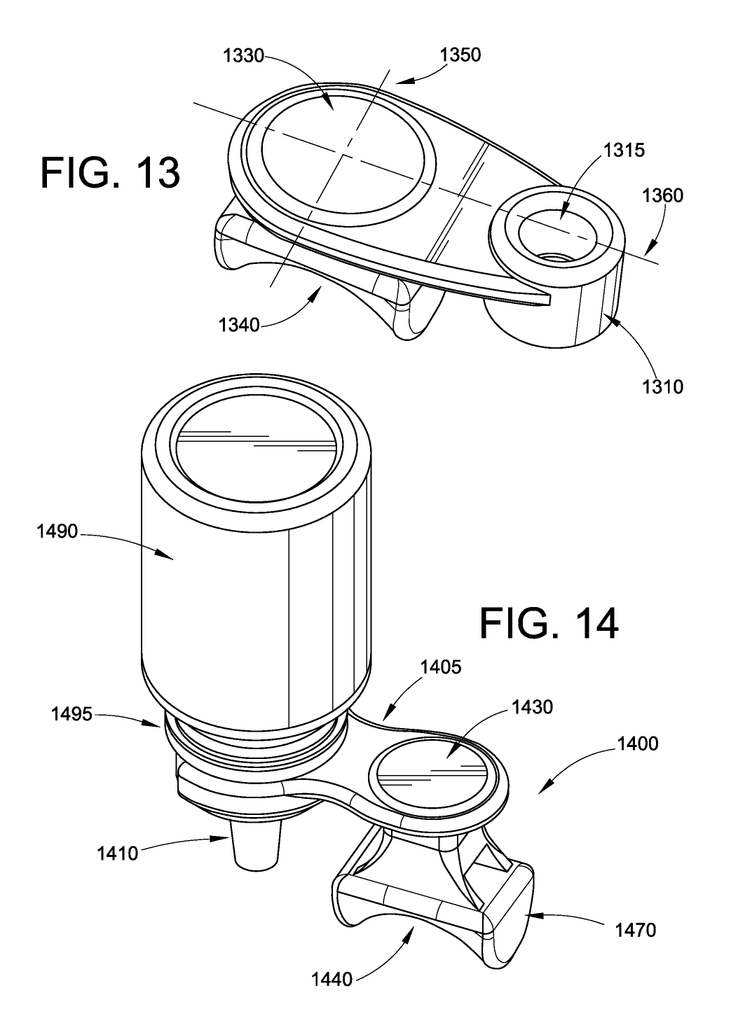

Referring now to FIG. 13, a single delivery well alternate embodiment of the eye dropper holder 1300 is presented with the stabilizing forehead pillars removed from the delivery platform 1305. This embodiment holds at least one delivery well 1310 to encompass the tip of the eye drop dispenser in place. The nasal bridge 1340 may be symmetric and is attached to the delivery platform 1305 substantially below a holding spot 1330 located within the center of the delivery platform 1305. The delivery well is of a shape and diameter to accommodate the shape of an eye drop dispenser tip. The device 1300 may be symmetric about the major axis 1360 but if the device contains only one delivery well 1310, then the device 1300 will not be symmetric about the minor axis 1350. The eye drop dispenser tip may be held in place by a variety of means including, but not limited to, a frictional surface, a threaded screw, a blade that extends from the delivery well and a bracket.

While the present embodiment is oval-shaped, other variants or embodiment of the device may include a delivery platform 1305 in a shape other than an ellipse including, but not limited to, square, rectangle, triangle, or other oblong shape. The delivery platform 1305 also may be asymmetrical to accommodate a user with eyes at different distances from the nose. The interior surface 1315 of the delivery well 1310 may also assume any form previously presented such as tapered, containing a blade, threaded sides and the like.

Referring now to FIG. 14, a single delivery well 1405 embodiment of the eye dropper holder 1400 is presented with the stabilizing forehead pillars removed from the delivery body. This embodiment holds the neck 1495 of the eye drop dispenser in place using a delivery well 1410. The delivery well 1410 may be tapered or may be brackets. The nasal bridge 1440 is attached to the delivery platform 1405 below the holding spot 1430 such that the sides of the nasal bridge 1470 do not contain nasal pads, but could contain nasal pads in an alternative embodiment. The eye drop dispenser 1490 may be held in place by a variety of means including, but not limited to, a frictional surface, a threaded screw, a blade that extends from the delivery well and the bracket described in FIG. 2.

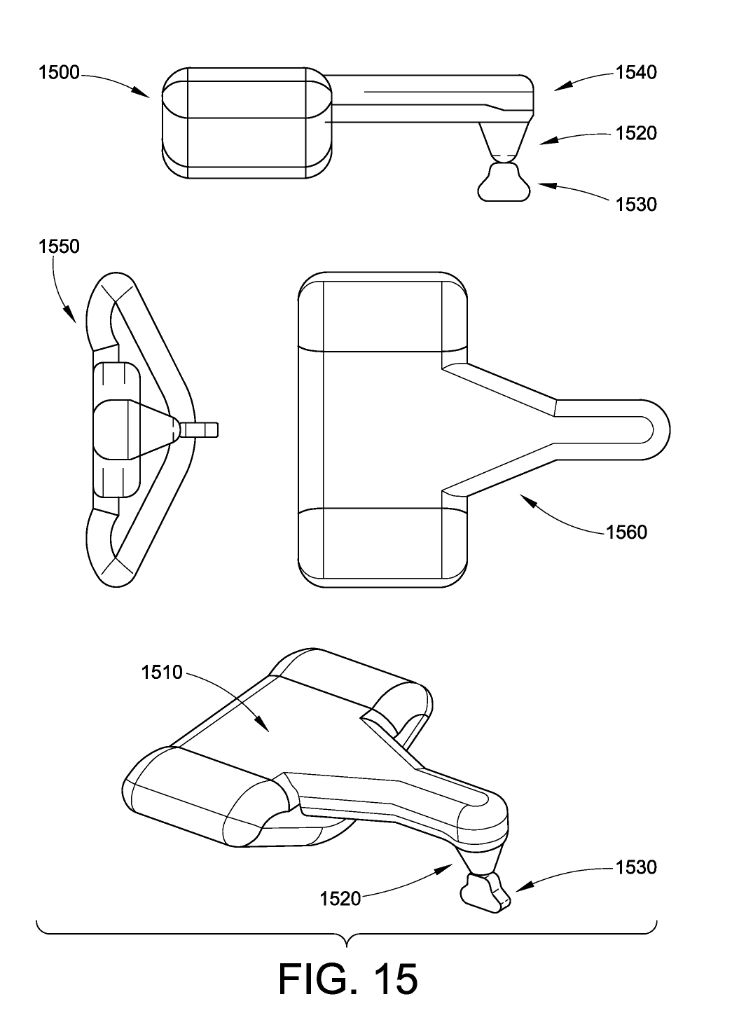

Referring now to FIG. 15, a disposable alternate embodiment of the eye dropper holder 1500 is presented. A self-contained eye drop reservoir 1510 is contained which holds a supply of eye drops within a hollow body of an eye drop dispenser. The device 1500 is contoured to fit across the user's nasion and lay on the nose as does the previously presented embodiments. The reservoir of drops 1510 are dispensed from the device through a portal 1520 which is blocked by a nib 1530 that plugs a drop dispensing portal 1520. The nib 1530 can be broken off in order to initiate the flow of eye drops through the portal and from the reservoir 1510. The disposable device is also presented in a side view 1540, in a frontal view 1550, and in a top view 1560.

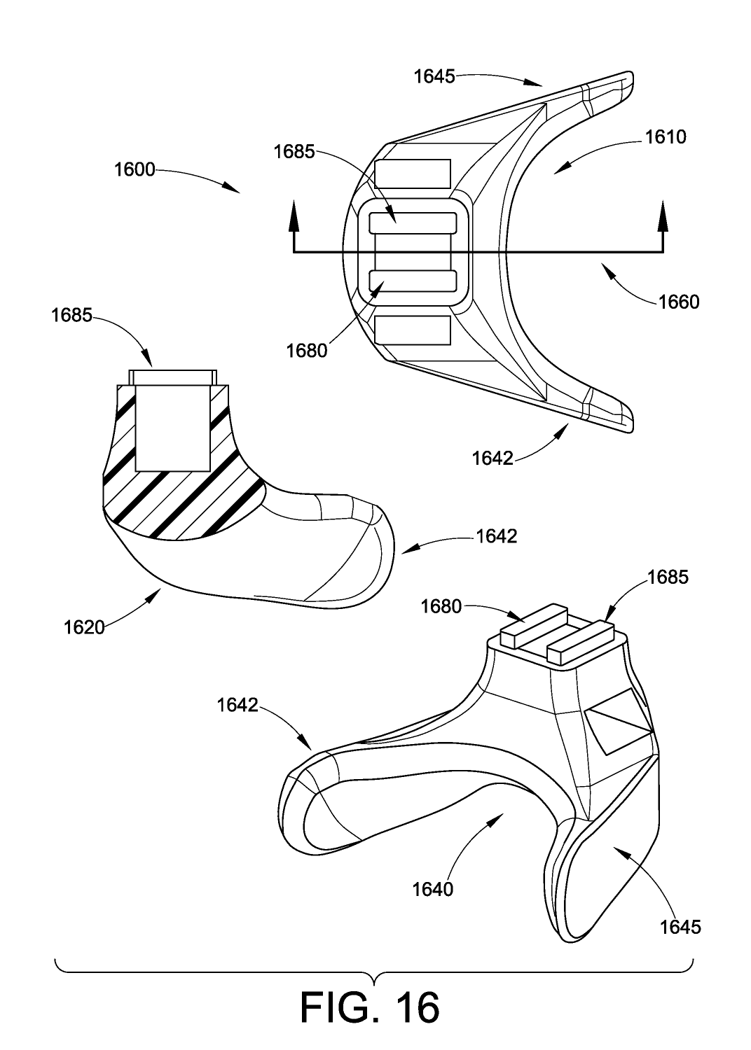

Referring now to FIG. 16, an alternate embodiment is presented 1600 where the nasal bridge 1640 attached to the underside of the eye dropper holder is asymmetric. The nasal bridge is asymmetric such that the nasal pads 1642, 1645 located on the lower contour of the nasal bridge 1640 are of different sizes, shapes, lengths, widths, heights, different distances from the delivery platform, or different distances from the axis 1660 between the center of the two attaching buttons 1680. 1685. Presented also is a top view 1610 and a side view 1620.

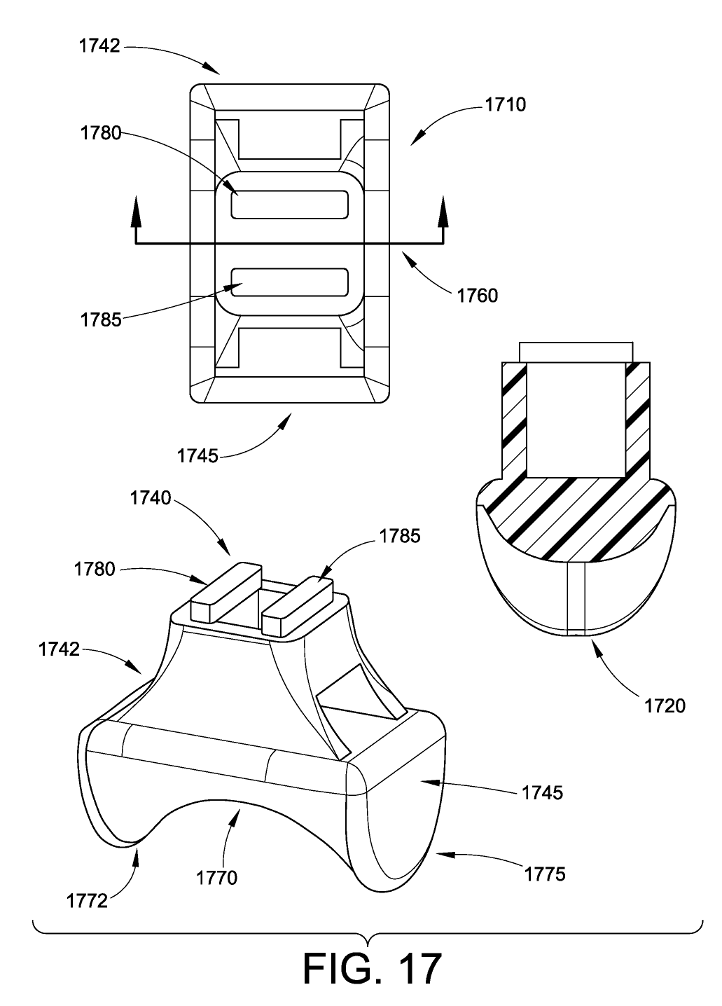

Referring now to FIG. 17, an alternate embodiment 1700 is presented where a symmetric nasal bridge 1740 does not contain nasal pads. Instead of nasal pads, the present embodiment of the nasal bridge 1740 contains a nasion bridge 1770 which cantilevers from the center of the nasion bridge 1770 in a gradual curve to meet 1772, 1775 two sides of the nasal bridge 1742, 1745. The nasion bridge fits against the user's nasion. The sides of the nasal bridge 1742, 1745 do not fit snuggly against the sides of the nose as do nasal pads. Rather the sides of the nasal bridge 1742, 1745 touch the sides of the upper bridge of the nose to prevent the nasal bridge from sliding off of the nose and prevent movement of the eye drop dispenser 1700 in a lateral axis of motion drawn between the two eyes. The top of the nasal bridge 1700 above the sides of the nasal bridge 1742, 1745 contains two tabs 1780, 1785 which extend upward as shafts. The tabs 1780, 1785 may be rectangular in shape with the longest dimension being parallel to the direction of the sides of the nasal bridge 1742, 1745. The tabs in the present embodiment are rectangular, but could easily work being of any geometric shape. The tabs 1780, 1785 may be used to attach the nasal bridge to the delivery platform by fitting into holes or other types of receptacles contained within the delivery platform for the purpose of receiving the tabs. The nasal bridge 1700 is symmetric about an axis 1760 between the two sides of the nasal bridge 1742, 1745 and between the tabs 1780, 1785. The nasal bridge 1700 may become permanently attached after being initially attached or may routinely be disengaged after each use in order to facilitate compact storage. Presented also is a top view 1710 and a side view 1720 of the nasal bridge.

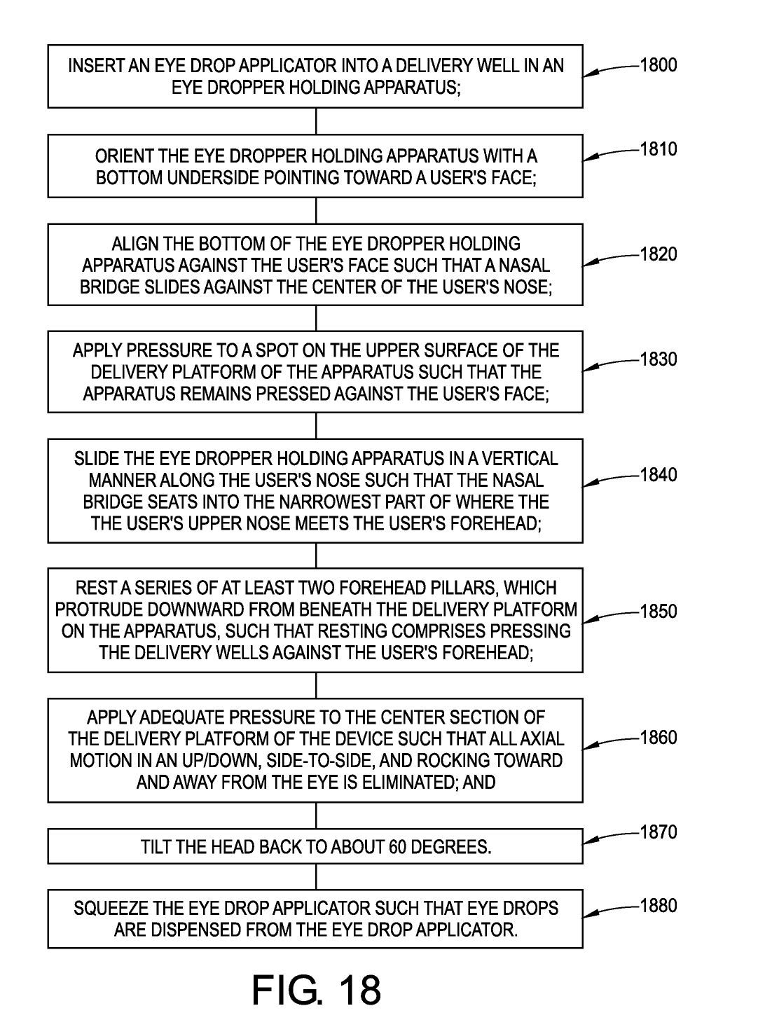

Referring now to FIG. 18, the method of using the device to apply eye drops is comprised of the following steps. First, the user inserts an eye drop application apparatus 1800, such as a bottle of eye drops or an eye dropper, into one or both of the delivery wells. The user then orients 1810 the eye dropper holding apparatus with a bottom underside pointing toward a user's face and with the eye drop supply on the side opposite the user's face side. The user then aligns 1820 the bottom of the eye dropper holding apparatus against the face so that a nasal bridge slides against the center of the user's nose and applies pressure to a spot on the center of the upper surface of the delivery platform of the apparatus such that the apparatus remains pressed against the user's face. The user then slides 1840 the eye dropper holding apparatus in a vertical manner along the user's nose such that the nasal bridge eventually automatically seats into the narrowest part of the user's nasion, where the user's upper nose meets the user's forehead. The user also rests 1850 the series of at least two forehead pillars, which protrude downward from beneath the delivery platform on the apparatus onto the user's forehead. The user should apply adequate pressure 1860 to the center section of the delivery platform of the device such that all axial motion in an up/down, side-to-side and rocking toward and away from the eye is eliminated. The user should tilt the head back 1870 so that the optimal angle between the user's head and the horizon is about 60 degrees. The user finally squeezes the eye drop applicator 1880 such that eye drops are dispensed from the eye drop applicator. The user may repeat this step as many times as necessary. The user may also perform these steps in an order different from the order described here, such as tilt the head back at a 60 degree angle as the first step or in between any of the two steps mentioned herein.

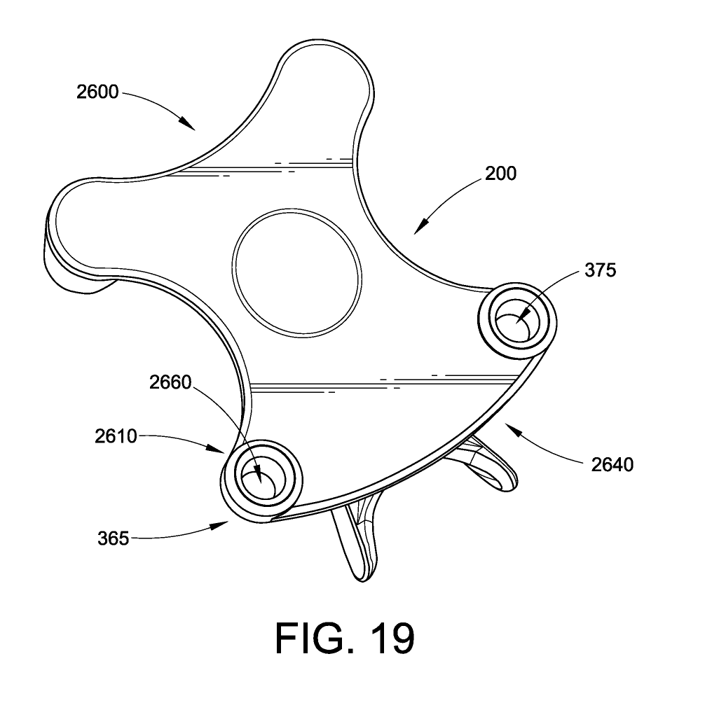

FIG. 19 presents an alternative embodiment of the present device 2600. The illustration presents a view of the delivery platform 200 with the two delivery wells 365, 375. The wells presented in this embodiment taper such that the upper opening 2610 is wider in diameter than the diameter of the lower opening 2660. This also could be presented as the upper diameter 2610 being the same or similar as the lower opening 2660. The alternative embodiment presented 2600 leads to the delivery well inner diameter protruding beneath the delivery well 200.

In a further embodiment, the edge 2640 of the main platform 200 is now flush into the outer portion of the deliver well 375.

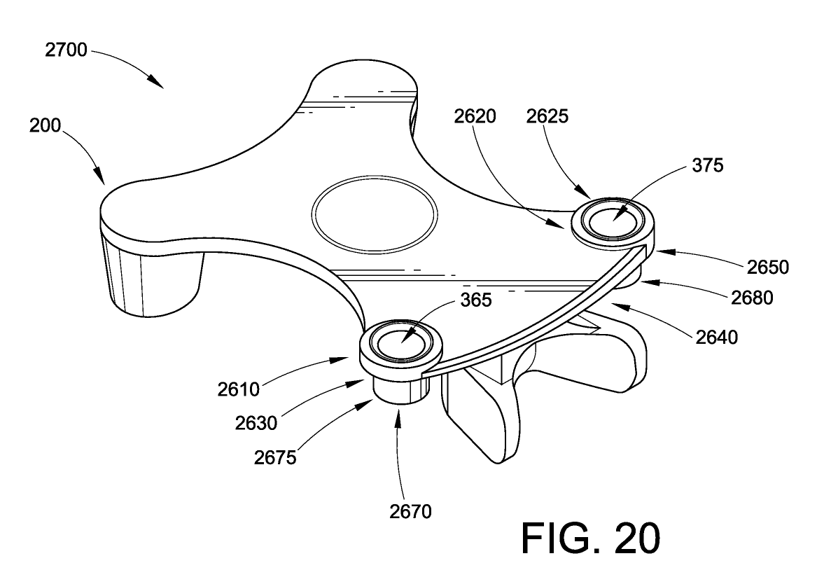

FIG. 20 presents another view of this embodiment 2700. Here the delivery platform 200 shows the delivery wells 365, 375 and also shows the side 2630 of the deliver well and the bottom portion 2670, 2680 of the delivery well 2630 protruding beneath the delivery platform 200. The top of the delivery well 2610 also protrudes at a substantial distance 2625 above the delivery platform 200 such that the top of the delivery well 2620 is not flush with the delivery platform 200, but rather is raised a distance above said delivery platform 2625. The delivery well may also be tapered 2680 such that the bottom diameter 2675 is smaller than the top opening.

The edge 2640 of the delivery platform 200 is also shown flush 2650 with the side of the delivery well 2620 such that the edge 2640 blends into 2650 the side of the delivery well 2620.

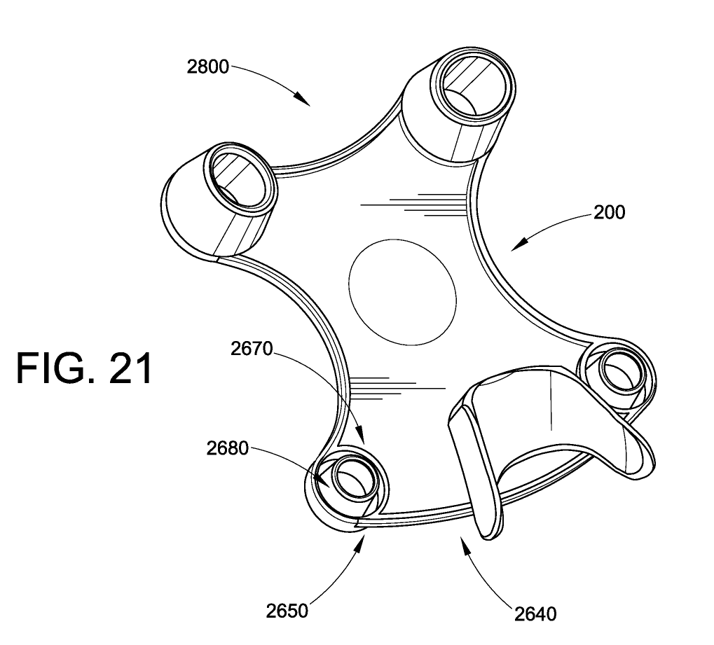

FIG. 21 presents a view of the same embodiment from below 2800. Here the delivery well 2680 is illustrated as protruding from beneath the delivery platform in a manner that the delivery well bottom portion is not flush with the bottom of the delivery platform 200 but rather is at a substantial distance 2080 below. The edge 2640 is also shown flush 2650 with the side of the delivery well. Diameter 2670 is also shown.

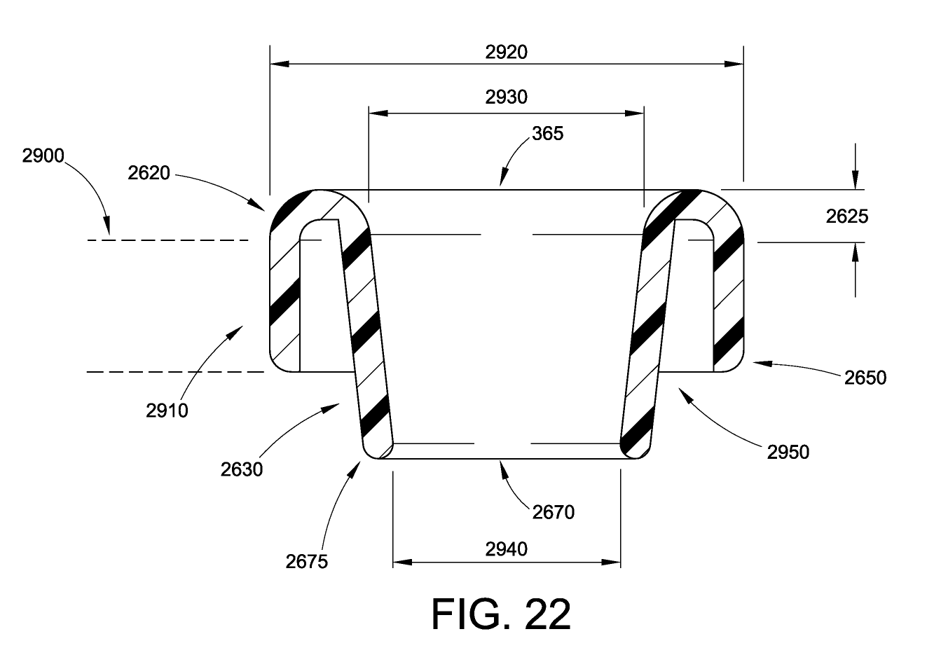

FIG. 22 presents a detailed and sectioned view of this embodiment of the delivery well 2900. Here the delivery well presented earlier in the application is shown in a cutaway view. Here the portion of the delivery well that protrudes above the delivery platform is illustrated as is the portion of the delivery well that protrudes beneath the delivery platform 2630. The outer diameter of the delivery well 2920 comprises the protrusion above 2620 the delivery platform. The inner diameter of the delivery well 2730 may be larger than the bottom diameter of the delivery well 2940 at the bottom portion of the delivery well 2670.

Furthermore, the edge of the delivery platform 2620 which is at the portion of the delivery platform that protrudes above the delivery platform 2620 is a distance 2625 above the main delivery platform 2910. The delivery well emerges from the bottom of the delivery platform 2910 at a point 2650 such that the portion 2950 of the delivery well is below the delivery platform. This section 2950 acts as an eyelash guard to prevent the eyelash from coming in contact with the tip of an eye drop dispenser. This serves to reduce contamination because the eyelash will impact this lower portion 2950 and deposit contaminants at the edge 2675 of the lower portion 2950, greatly reducing the impact of an eyelash against the tip of an eye drop dispenser and preventing eyelash contaminants from being deposited on an eye drop dispenser.

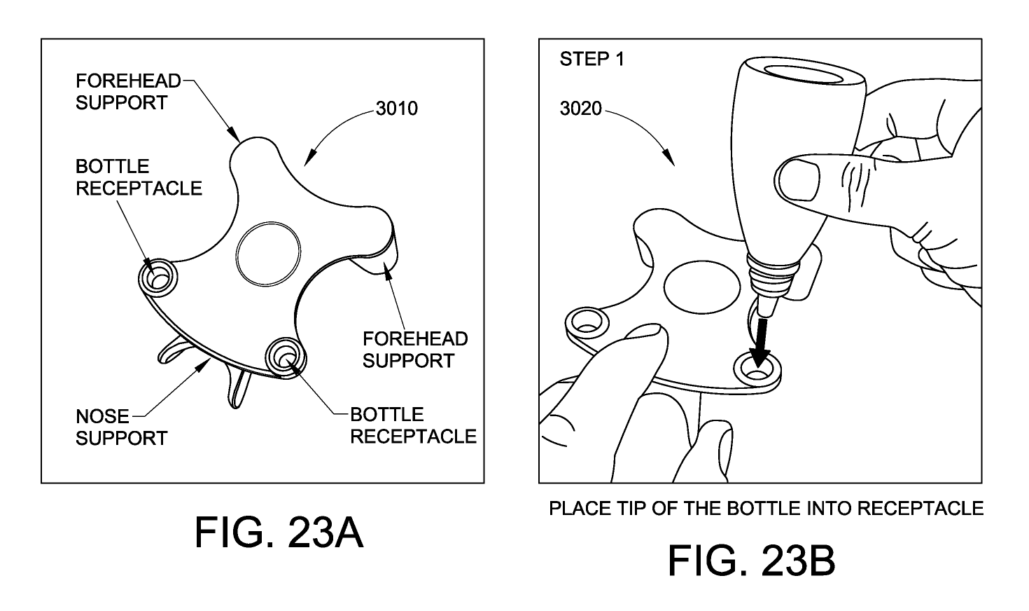

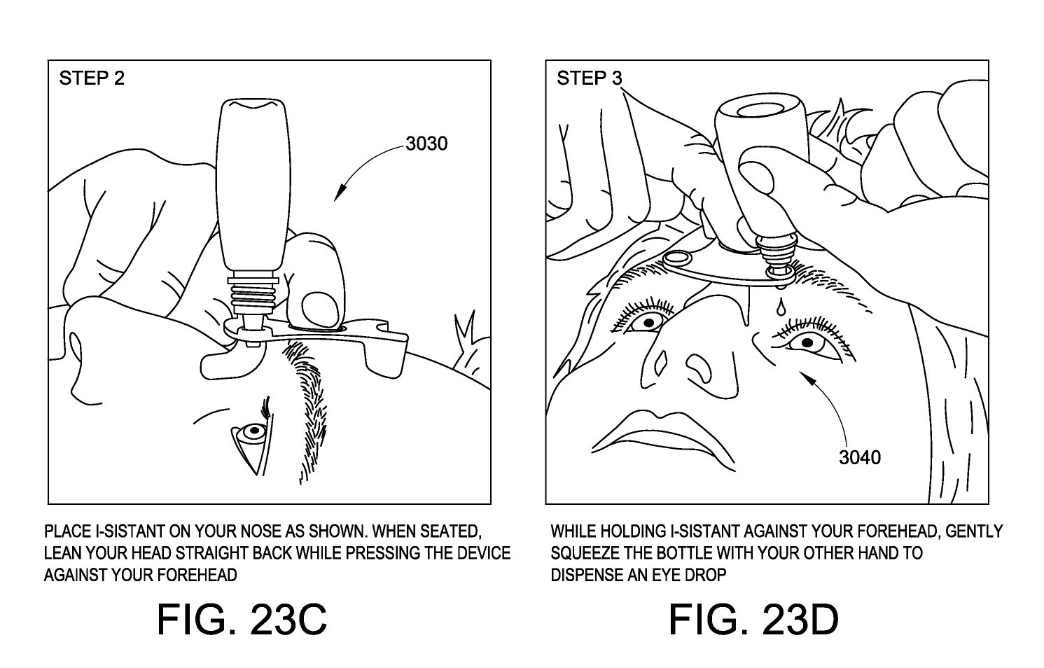

FIGS. 23A-D present a series of instructions for the use of the device. FIG. 23A illustrates device elements 3010. In FIG. 23B, the method 3020 of inserting the eye drop dispenser into a bottle receptacle is identified in step 1. FIG. 23C is Step 2 showing 3030 how to hold the device during use. FIG. 23D is Step 3 showing 3040 how to apply an eye drop.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

What is claimed is:

1. A condensed eye dropper alignment apparatus comprising:
a delivery platform with a front side and a back side, both sides sharing a common upper edge and a common lower edge;
a pair of delivery wells located at opposite ends of a delivery platform, the delivery wells opening to facilitate the insertion of an eye drop dispensing apparatus, the depth wise dimension of the delivery wells directed perpendicular to the plane of the delivery platform and the bottom of the delivery well extending below the delivery platform; and
a nasal bridge extending from the back side of the delivery platform, wherein the nasal bridge is in a plane parallel to the delivery platform, the arc of the nasal bridge being sized to dispose into a snug cradle position across the lowest point of the user's nasion, and the ends of the nasal bridge cantilever to form nasal pads which straddle the width of a human user's nose.

2. The apparatus of claim 1, wherein the delivery wells accommodate the neck of an eye drop source.

3. The apparatus of claim 1, wherein the delivery wells accommodate the tip of an eye drop dispensing source.

4. The apparatus of claim 1, wherein the delivery wells retain the eye drop dispenser by at least one of screw threads, frictional surface and a blade that protrudes from the delivery well.

5. The apparatus of claim 1, wherein the nasal pads are of different dimensions.

6. A condensed eye dropper alignment apparatus comprising:
a delivery platform with a front side and a back side, both sides sharing a common upper edge and a common lower edge:
a pair of circular delivery wells located at opposite ends of a delivery platform, the circular delivery wells comprised of an upper bracket and a lower bracket between which an eye drop dispensing apparatus may be inserted and held in a manner that prevents movement; and
a nasal bridge extending from the back side of the delivery platform, wherein the nasal bridge is in a plane parallel to the delivery platform, the arc of the nasal bridge being sized to dispose into a snug cradle position across the lowest point of the user's nasion, and the ends of the nasal bridge cantilever to straddle the width of a human user's nose.

7. The apparatus of claim 6 further comprising the upper and lower brackets being of the same length.

8. The apparatus of claim 6 further comprising the upper and lower brackets being of different lengths.

9. The apparatus of claim 6 further comprising the brackets being flexible such that an eye drop dispenser may be inserted through a gap between the upper bracket and the lower bracket.

10. The apparatus of claim 6, wherein the nasal pads are of different dimensions.

11. A condensed eye dropper alignment apparatus comprising
a delivery platform with a front side and a back side, both sharing a common upper edge and a common lower edge;
a single delivery well open to facilitate the insertion of an eye drop dispensing apparatus; and
a nasal bridge extending from the back side of the delivery platform, wherein the nasal bridge is in a plane parallel to the delivery platform, the arc of the nasal bridge being sized to dispose into a snug cradle position across the lowest point of the user's nasion, and the ends of the nasal bridge cantilever to straddle the width of a human user's nose.

12. The apparatus of claim 9, wherein the delivery well accommodates the neck of an eye drop source.

13. The apparatus of claim 9, wherein the delivery well accommodates the tip of an eye drop dispensing source.

14. The apparatus of claim 9, wherein the delivery well retains the eye drop dispenser by at least one of screw threads, frictional surface and a blade that protrudes from the delivery well.

15. The apparatus of claim 9, wherein the delivery well is comprised of an open ended upper bracket and a lower bracket between which an eye drop dispensing apparatus may be inserted and held in a manner that prevents movement of the eye drop dispenser.

16. The apparatus of claim 9 further comprising the brackets being flexible such that an eye drop dispenser may be inserted through a gap between the upper bracket and the lower bracket.

17. A disposable eye dropper dispenser and alignment apparatus comprising:
a hollow delivery platform contoured to conform to the edge of a user's nose including a lowest point of the user's nasion,
an internal reservoir for containing eye drop liquid;
a single delivery portal to facilitate the dispensing of eye drops; and
a removable nib attached to plug the end of the portal and prevent eye drop liquid from flowing out of the portal.

18. An eye dropper alignment apparatus comprising:
a delivery platform with a front side and a back side, both sides sharing an upper edge and a lower edge;
at least two supporting stabilizing forehead pillars extending perpendicular from the back side of the delivery platform and located on corners aligned with the upper edge of the delivery platform;
at least one delivery well opening to facilitate the insertion of an eye drop dispensing apparatus, the depth wise dimension of the at least one delivery well directed perpendicular to the plane of the delivery platform and parallel to the two supporting forehead pillars, the at least one delivery well located in a corner aligned with the lower edge opposite of the upper edge and the bottom of the delivery well extending below the bottom of the delivery platform; and
a nasal bridge extending from the back side of the delivery platform, wherein the nasal bridge is in a plane parallel to the delivery platform, the arc of the nasal bridge being sized to dispose into a snug cradle position across the lowest point of the user's nasion, and the ends of the nasal bridge cantilever to straddle the width of a human user's nose, wherein the cantilevers are of different lengths.

19. An eye dropper alignment apparatus comprising:
a delivery platform with a front side and a back side, both sides sharing an upper edge and a lower edge;
at least two supporting stabilizing forehead pillars extending perpendicular from the back side of the delivery platform and located on corners aligned with the upper edge of the delivery platform;
at least one delivery well opening to facilitate the insertion of an eye drop dispensing apparatus, the depth wise dimension of the at least one delivery well directed perpendicular to the plane of the delivery platform and parallel to the two supporting forehead pillars, the at least one delivery well located in a corner aligned with the lower edge opposite of the upper edge and the bottom of the delivery well extending below the delivery platform; and
a nasal bridge extending from the back side of the delivery platform, wherein the nasal bridge is in a plane parallel to the delivery platform, the arc of the nasal bridge being sized to dispose into a snug cradle position across the lowest point of the user's nasion, and the ends of the nasal bridge blend into the sides of the nasal bridge and are truncated to prevent movement in a lateral axis.

20. A method of applying eye drops using a locating and stabilization system comprising the steps of:
inserting an eye drop applicator into a delivery well in an eye dropper holding apparatus;
orienting the eye dropper holding apparatus with a bottom underside pointing toward a user's face;
aligning the bottom of the eye dropper holding apparatus against the user's face such that a nasal bridge slides against the center of the user's nose;
applying pressure to a spot on the upper surface of the delivery platform of the apparatus such that the apparatus remains pressed against the user's face;
sliding the eye dropper holding apparatus in a vertical manner along the user's nose such that the nasal bridge seats into the narrowest part of where the user's upper nose meets the user's forehead;
resting a series of at least two forehead pillars, which protrude downward from beneath the delivery platform on the apparatus, such that resting comprises pressing the delivery wells against the user's forehead;
applying adequate pressure to the center section of the delivery platform of the device such that all axial motion in an up/down, side-to-side, and rocking toward and away from the eye is eliminated; and
squeezing the eye drop applicator such that eye drops are dispensed from the eye drop applicator.
